(19) United States
(12) Patent Application Publication
(10) Pub. No.: US 2026/0027559 A1
(43) Pub. Date: Jan. 29, 2026

(54) PROCESS FOR PREPARING, BY HYDROFORMYLATION, SHORT-CHAIN OLEFINS IN THE GAS PHASE

(71) Applicant: Evonik Oxeno GmbH & Co. KG, Marl (DE)

(72) Inventors: Robert Franke, Marl (DE); Linda Arsenjuk, Dortmund (DE); Vinzenz Fleischer, Marl (DE); Corina Nentwich, Datteln (DE); Frank Stenger, Alzenau (DE); Marc Oliver Kristen, Haltern am See (DE); Peter Kreis, Dortmund (DE)

(73) Assignee: Evonik Oxeno GmbH & Co. KG, Marl (DE)

(21) Appl. No.: 18/995,401

(22) PCT Filed: Apr. 26, 2023
(86) PCT No.: PCT/EP2023/060906

§ 371 Date: Jan. 16, 2025

(30) Foreign Application Priority Data
Jul. 19, 2022 (EP) 22185748.5

(51) Int. Cl. B01J 31/24; B01J 21/18; B01J 31/02; C07C 45/50

(52) U.S. Cl. CPC B01J 31/24; B01J 21/18; B01J 31/0235; C07C 45/50; B01J 2231/321; B01J 2523/822; B01J 2531/822

(57) ABSTRACT

The invention relates to a process for hydroformylating short-chain olefins, especially C2 to C8 olefins, in which the catalyst system, which comprises a metal from group 8 or group 9 of the periodic table of the elements, at least one organic phosphorus-containing ligand and a stabilizer, is present in a heterogenized form on a support composed of a porous ceramic material, the support consisting of a bed of shaped SiC bodies in which the proportion of pores having a pore diameter in the range between 10 nm to 1000 nm, based on the total amount of pores in the shaped SiC bodies, is 20 to 70%, preferably 25 to 50%.

The project that resulted in this patent application was supported under grant No. 869896 of the European Union's Horizon 2020 research and innovation program.

The present invention relates to a process for hydroformylating short-chain olefins, especially C2 to C8 olefins, in which the catalyst system, which comprises a metal from group 8 or group 9 of the periodic table of the elements, at least one organic phosphorus-containing ligand and a stabilizer, is present in a heterogenized form on a support composed of a porous ceramic material, the support consisting of a bed of shaped SiC bodies in which the proportion of pores having a pore diameter in the range between 10 nm to 1000 nm, based on the total amount of pores in the shaped SiC bodies, is 20 to 70%, preferably 25 to 50%.

Hydroformylation is one of the most important reactions in industrial chemistry, having an annual global production capacity of several million tonnes. It involves reacting alkenes (olefins) with a mixture of carbon monoxide and hydrogen (also: synthesis gas or syngas) using a catalyst to give aldehydes, which are important and valuable intermediates in the production of chemical bulk products such as alcohols, esters or plasticizers.

Hydroformylation is on the industrial scale carried out exclusively under homogeneous catalysis. The soluble transition metal catalyst systems are typically based on cobalt or rhodium, which is often used together with phosphorus-containing ligands, for example phosphines or phosphites, for the hydroformylation of comparatively short-chain olefins.

There are various problems with the known processes, these problems being linked in particular to the fact that both rhodium and cobalt and compounds thereof are comparatively costly. Large amounts of energy and outlay on process are necessary in order for catalyst losses during the hydroformylation process to be avoided as far as possible, for example through catalyst recycling steps, some of which are very laborious. Moreover, product purification steps are becoming more laborious in order to ensure that as far as possible no catalyst residues remain in the product.

Further problems with the known homogeneously catalysed processes are the stability of the ligands, which have to withstand the conditions of the hydroformylation, such as temperature, pressure, pH etc., and the consumption during the process of the solvent used, which has to be compensated for by replenishment.

In order to get around the abovementioned problems in homogeneously catalysed hydroformylation, hydroformylation processes have been developed in which the catalyst system is heterogenized, especially by immobilization on a support material (cf. introductory discussion in WO 2015/028284 A1). The terms “heterogenization” and “immobilization” should accordingly be understood as meaning that the catalyst is immobilized through the formation of a thin liquid film with the aid of an ionic liquid on the surface and/or in the pores of a solid support material and that there is no reaction solution in the conventional sense in which the catalyst is homogeneously dissolved.

Hydroformylation processes in which the catalyst is present on a support material in a heterogenized form are disclosed for example in WO 2015/028284 A1, EP 3 632 885 A1, EP 3 744 707 A1, EP 3 632 886 A1 or in EP 3 736 258 A1.

The problem with the known heterogenized catalyst systems is that the supports used for this purpose onto which the catalyst is applied are not commercially available or are available only with difficulty. Instead, such a support, for example a monolithic block in which channels may optionally still be present, must be specifically and laboriously produced solely for this purpose. This not only increases costs, but also carries the risk that the support cannot be obtained and used immediately. This can lead to production losses. Furthermore, the size of the monolithic structures is limited by their mechanical stability, which places limits on reactor design.

The object of the present invention was therefore to provide a process for hydroformylating olefins that does not have the abovementioned problems. More particularly, a support should be provided that is easy to obtain and does not need to be produced specifically for this purpose.

This object is achieved according to claim 1 in that the hydroformylation employs a catalyst system wherein catalyst system in a heterogenized form on a support that consists of a bed of shaped SiC bodies in which the proportion of pores having a pore diameter in the range between 10 nm to 1000 nm, based on the total amount of pores in the shaped SiC bodies, is 20 to 70%, preferably 25 to 50%.

The present invention thus provides a process for hydroformylating C2 to C8 olefins in a reaction zone using a heterogenized catalyst system, wherein the process is characterized in that
a gaseous feed mixture containing the C2 to C8 olefins is passed together with synthesis gas over a support composed of a porous ceramic material on which the catalyst system, which comprises a metal from group 8 or group 9 of the periodic table of the elements, at least one organic phosphorus-containing ligand and a stabilizer, is present in a heterogenized form; and
the support consists of a bed of shaped SiC bodies in which the proportion of pores having a pore diameter in the range between 10 nm to 1000 nm, based on the total amount of pores in the shaped SiC bodies, is 20 to 70%, preferably 25 to 50%.

The feed mixture used may be any mixture comprising C2 to C8 olefins, preferably C2 to C5 olefins, especially ethene, propene, 1-butene, 2-butene, 1-pentene or 2-pentene, as reactants. The amount of olefins in the feed mixtures should understandably be high enough to be able to economically operate a hydroformylation reaction. The feed mixtures that may be employed in the process according to the invention include in particular also technical mixtures from the petrochemical industry, for example raffinate streams (raffinate I, II or III) or crude butane. According to the present invention, crude butane comprises 5% to 40% by weight of butenes, preferably 20% to 40% by weight of butenes (the butenes are composed of 1% to 20% by weight of 1-butene and 80% to 99% by weight of 2-butene), and 60% to 95% by weight of butanes, preferably 60% to 80% by weight of butanes.

The process according to the invention is carried out in at least one reactor in which the hydroformylation according to the invention takes place. The support with the heterogenized catalyst system is arranged in the at least one reactor. In a further embodiment of the present invention, the process can also be carried out in a plurality of reactors, which may be connected in parallel or in series. Preferably, the reactors are in the present case connected in parallel and are used alternately. An example of a suitable reactor type are conventional shell-and-tube reactors.

The hydroformylation is preferably carried out under the following conditions: The temperature in the hydroformylation should be in the range from 65 to 200° C., preferably 75 to 175° C. and more preferably 85 to 150° C. The temperature may be set by means of a suitable cooling apparatus, for example a cooling jacket. The pressure during the hydroformylation should not exceed 35 bar, preferably 30 bar, more preferably 25 bar. The molar ratio between synthesis gas and the feed mixture should be between 6:1 and 1:1, preferably between 5:1 and 3:1. Optionally, the feed mixture can be diluted with inert gas, for example with alkanes present in technical hydrocarbon streams.

The catalyst system used in the hydroformylation process according to the invention preferably comprises a transition metal from group 8 or 9 of the periodic table of the elements, especially iron, ruthenium, iridium, cobalt or rhodium, further preferably cobalt or rhodium, particularly preferably at least one organic phosphorus-containing ligand and a stabilizer.

The stabilizer is preferably an organic amine compound, more preferably an organic amine compound containing at least one 2,2,6,6-tetramethylpiperidine unit of formula (I):

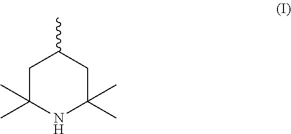

In a particularly preferred embodiment of the present invention, the stabilizer is selected from the group consisting of the compounds of the following formulas (I.1), (I.2), (I.3), (I.4), (I.5), (I.6), (I.7) and (I.8)

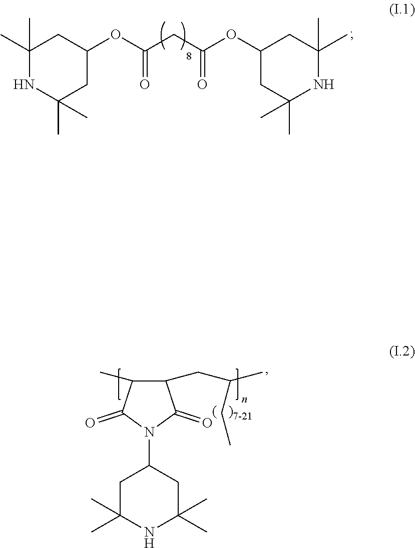

where n is an integer from 1 to 20;

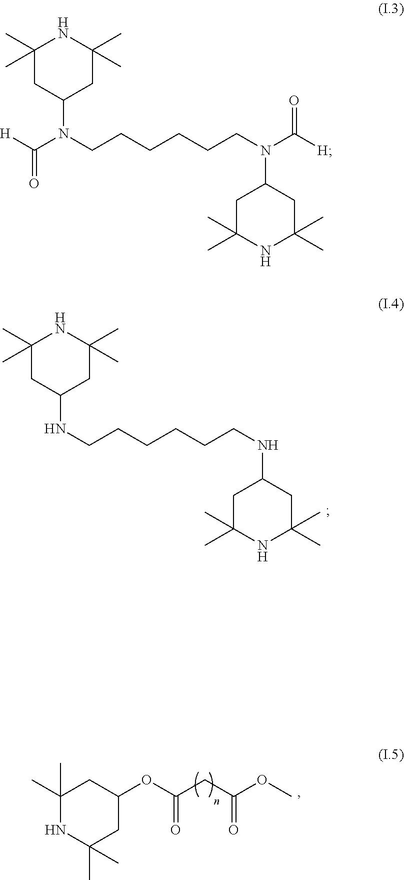

where n is an integer from 1 to 17;

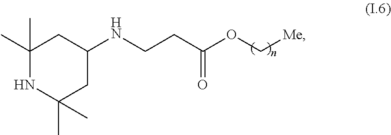

where n is an integer from 1 to 17;

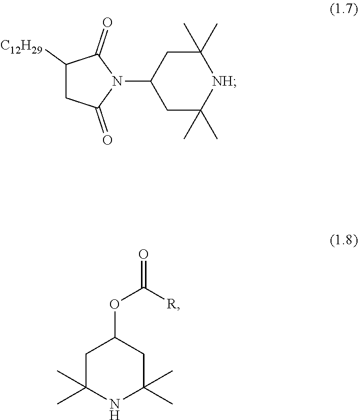

where R is a C6 to C20 alkyl group.

For all film-forming components, i.e. in this case the stabilizer, the gas solubility for the reactants should be better than the gas solubility of the products. In that way alone, it is possible to achieve partial physical separation between reactant olefins used and product aldehydes formed. In principle, other film-forming substances would also be conceivable for the purpose, but care should be taken to ensure there is no increased formation of high boilers and/or that the resupply of the reactant olefins is limited.

The organic phosphorus-containing ligand selected for the catalyst system according to the invention may be any ligand known for the hydroformylation. A large number of suitable ligands is known from the patent and specialist literature to those skilled in the art, for example mono- or biphosphite ligands. The organic phosphorus-containing ligand preferably has a biphosphite structure according to the general formula (II)

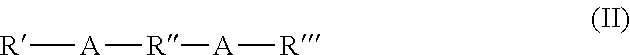

where R′, R″ and R′″ are each organic radicals and the two A are each a bridging —O—P(—O)₂— group, wherein two of the three oxygen atoms —O— are attached respectively to the radical R′ and to the radical R′″, with the proviso that R and R are nonidentical, The organic radicals R′, R″ and R″″ preferably do not contain any terminal trialkoxysilane groups.

In a preferred embodiment, R′, R″ and R′″ in the compound of the formula (VI) are preferably selected from substituted or unsubstituted 1,1′-biphenyl, 1,1′-binaphthyl and ortho-phenyl groups, especially from substituted or unsubstituted 1,1′-biphenyl groups, with the proviso that R′ and R′″ are nonidentical. More preferably, the substituted 1,1′-biphenyl groups have an alkyl group and/or an alkoxy group in the 3,3′ and/or 5,5′ positions of the 1,1′-biphenyl base structure, especially a C1-C4 alkyl group, more preferably a tert-butyl and/or methyl group, and/or preferably a C1-C5 alkoxy group, more preferably a methoxy group.

The abovementioned catalyst system is according to the invention present in a heterogenized form on a support composed of a porous ceramic material. In the context of the present invention, the expression “in a heterogenized form on a support” is understood to mean that the catalyst system is immobilized on the inner and/or outer surface of a solid support material through the formation of a thin, solid or liquid film with the aid of the stabilizer. The film may also be solid at room temperature and liquid under reaction conditions.

The inner surface of the solid support material comprises more particularly the inner surface area of the pores. The concept of immobilization includes both the situation where the catalyst system and/or the catalytically active species is present in dissolved form in the solid or liquid film and the situation where the stabilizer acts as an adhesion promoter or where the catalyst system is adsorbed on the surface, but not chemically/covalently bonded to the surface.

According to the invention, there is thus no reaction solution in the conventional sense in which the catalyst is homogeneously dissolved; instead, the catalyst system is dispersed on the surface and/or in the pores of the support.

The support according to the present invention onto which the catalyst system is applied consists of silicon carbide (SiC). The support further consists of a bed of shaped SiC bodies in which the proportion of pores having a pore diameter in the range between 10 nm to 1000 nm, based on the total amount of pores in the shaped SiC bodies, is 20 to 70%, preferably 25 to 50%. Shaped bodies of this kind may be of various shapes, for example in the form of trilobes, rings, spheres, cylinders or the like. The size of the shaped SiC bodies may here be from 0.1 mm to 50 mm. Such shaped bodies are commercially available and may be produced for example by extrusion.

For the production of the support, older patent applications describe the application of a washcoat. This additional step can be dispensed with in the context of the present invention. Thus, no washcoat is applied, i e. the shaped SiC bodies do not have a washcoat. The catalyst system is applied onto the support, i.e. onto the shaped SiC bodies. For this purpose, a catalyst solution is first produced by mixing, especially at room temperature and ambient pressure, the catalyst solution comprising at least one organic phosphorus-containing ligand, at least one metal precursor, for example chlorides, oxides, carboxylates of the respective metal, at least one stabilizer and at least one solvent. It is optionally possible to use an ionic liquid in the production of the catalyst system, but the catalyst solution can also explicitly be prepared without ionic liquid. The catalyst solution should especially be produced in an inert environment, for example in a glovebox “Inert environment” in this case means an atmosphere that is as far as possible free of water and oxygen.

The solvent may be chosen from all solvent classes (protic, aprotic, polar or nonpolar). A prerequisite for the solvent is the solubility of catalyst system (ligand, metal precursor, stabilizer and optionally the ionic liquid) and preferably also of the high boilers formed in the hydroformylation. Solubility can be increased during the immobilization step by heating.

The solvent is preferably aprotic and polar, for example acetonitrile and ethyl acetate, or else aprotic and nonpolar, for example THF and diethyl ether. It is also possible to use as solvent chlorinated hydrocarbons, for example dichloromethane, or aldehydes.

The catalyst solution thus produced is then contacted with the support (optionally including washcoat), for example by dipping (dip-coating) or by filling a pressure vessel, for example directly in the reactor (in-situ impregnation). If the catalyst solution is applied outside the reactor, the support must of course be reinstalled in the reactor after the solvent has been removed. Preferably, the catalyst solution is applied to the support with the washcoat directly in the reactor because this can avoid potentially time-consuming installation and deinstallation steps and potential contamination of the catalyst.

In the case of in situ-impregnation, the reactor can prior to filling be purged with an inert gas, for example a noble gas, alkanes or nitrogen. The purging can be carried out at 1 to 25 bar, preferably under a slight positive pressure of 20 to 90 mbar, more preferably 30 to 60 mbar, above standard pressure. Before the purging with inert gas, the reactor can be cooled in order to prevent the solvent in the catalyst solution to be introduced from evaporating immediately. However, if the solvent has a boiling temperature greater than the reactor temperature, the cooling of the reactor can be dispensed with.

The reactor can be filled with the catalyst solution via the normal inlets/outlets, for example by means of a pump. Liquid distributors or nozzles within the reactor can ensure homogeneous distribution of the catalyst liquid, as can pressure drop internals or regulators for the metering rate that are optionally present.

After the catalyst system has been applied, the solvent is removed. This involves firstly discharging the residual catalyst solution via the reactor outlet. Solvent residues remaining in the reactor are then evaporated by adjusting the pressure or increasing the temperature. In another embodiment it is also possible for the pressure to be adjusted in tandem with an increase in temperature. The temperature may be 20 to 150° C., depending on the solvent. The pressure may be adjusted to a high vacuum (10⁻³ to 10⁻⁷ mbar), depending on the solvent, but overpressures of a few mbar up to several bar are also conceivable, depending on the solvent and temperature.

The stabilizer remains with the catalyst formed from the transition metal, especially cobalt or rhodium, and the organic phosphorus-containing ligand, in heterogenized form on the support.

The catalyst system can be applied to the support either directly in the reactor (in situ) or outside the reactor. If the catalyst system is applied outside the reactor, the support must always be transported with exclusion of air, which can be achieved for example with a nitrogen countercurrent. In a preferred embodiment of the present invention, the catalyst system is applied directly in the reactor, i.e. in situ. After the solvent has been removed, the reactor can be used immediately and charged with the feed mixture. This has the advantage that no time-consuming installation and deinstallation steps that would result in a prolonged reactor shutdown are needed. Moreover, this then no longer gives rise to any limitation on the size of the support, in that suitable spaces having inert environments are available in a particular size. The size of the support can be chosen freely according to the reactor design.

Once the catalyst system has been applied to the support and the solvent has been removed, the plant, more particularly the reactor, can be started up, i.e. put into operation, through a two-stage or multistage startup procedure. A suitable startup procedure is described for example in EP 3 632 887.

A gaseous output comprising at least a portion of the product aldehydes formed and at least a portion of the unreacted olefins is preferably withdrawn continuously from the reaction zone in which the hydroformylation according to the invention is carried out. The gaseous output may be subjected to one or more physical separation step(s) in which the gaseous output is separated into at least one phase rich in unreacted olefins and at least one phase rich in product aldehyde.

The physical separation can be carried out by known physical separation methods such as condensation, distillation, centrifugation, nanofiltration or a combination of two or more thereof, preferably condensation or distillation.

In the case of a multistage physical separation, the phase rich in product aldehyde that is formed in the first physical separation is sent to a second physical separation, especially a downstream removal of aldehyde, in which the product aldehyde is separated from the other substances present in this phase, commonly alkanes and reactant olefins. The phase rich in unreacted olefin can be recycled to the hydroformylation step or, in the case of a multistage configuration, to one of the hydroformylation steps in order to further hydroformylate the olefins present therein to the product aldehyde.

In the physical separation, in addition to the phases mentioned it is also possible to withdraw a purge gas stream having a composition identical or at least similar to the phase rich in unreacted olefin. The purge gas stream can likewise be conveyed to the second physical separation or aldehyde removal in order to remove the product aldehydes present therein and to discharge impurities (for example nitrogen in the synthesis gas) or inert substances (for example alkanes in the feed mixture) from the system. The impurities or inert substances can typically be removed in the second physical separation as volatile substances, for example at the top of a column.

The present invention also further provides a plant with which the present process can be carried out and that especially comprises a reactor in which the hydroformylation step according to the invention is carried out. In addition, the plant may comprise a physical separation unit with which the gaseous output from the hydroformylation step is separated into at least one phase rich in unreacted olefin and at least one phase rich in product aldehyde, this physical separation unit being arranged downstream of the hydroformylation according to the invention. Present downstream of the first physical separation there may be a second physical separation unit, especially an aldehyde removal unit, with which the product aldehyde is removed.

Even without further elaboration it is assumed that those skilled in the art will be able to utilize the description above to the greatest possible extent. The preferred embodiments and examples are therefore to be interpreted merely as a descriptive disclosure which is by no means limiting in any way whatsoever.

The present invention is more particularly elucidated hereinbelow with reference to examples. Alternative embodiments of the present invention are obtainable by analogy therewith.

### EXAMPLES

### Example 1

The support used was a commercially available SiC3-E2-HP SiC extrudate from Sicat Sard. The support was then introduced into a round reactor sleeve 20 cm in length and having a diameter of one inch (approx. 2.54 cm), with glass beads of similar size introduced above and below the granular material. The support was then loaded in situ (in the reactor) with a catalyst solution containing Rh(acac)(CO)₂, bisphephos (ligand), bis(2,2,6,6-tetramethyl-4-piperidyl)sebacate (stabilizer) and dichloromethane as solvent. This was done, after first purging the reactor with nitrogen, by introducing the catalyst solution into the reactor under a slight overpressure. After removal of the solvent from the reactor by discharge and evaporation, the hydroformylation was carried out.

The feed mixture used was a hydrocarbon stream having the following composition:

| | Amount |
|---|---|
| | (% by wt.) |
| 1-Butene/isobutene | 1 |
| cis-2-Butene | 4 |
| trans-2-Butene | 12 |
| n-Butane | 72 |
| Isobutane | traces |
| 2-Methylbutane | traces |

For the hydroformylation, the feed mixture was passed into the reactor together with synthesis gas (molar ratio of synthesis gas to feed mixture=3.5:1) at a total mass flow of 12.8 g/h. The hydroformylation was carried out at a temperature of 120-130° C. and a pressure of 10-17 bar. The total conversion of butenes (i.e. the conversion of all butenes present in the feed mixture), the proportion of high boilers and the n/iso selectivity (ratio of linear to branched products) was determined by gas chromatography via the product composition.

After an experiment duration of 250 hours, total conversion of butenes was 51% and the n/iso selectivity 95%. The proportion of high boilers was max. 0.04% by weight.

### Example 2

The procedure was as described in example 1. The support used was however a commercially available SiC4-E2-HP SiC extrudate from Sicat Sarl.

The feed mixture used was a hydrocarbon stream having the following composition:

| | Amount |
|---|---|
| | (% by wt.) |
| 1-Butene/isobutene | 1 |
| cis-2-Butene | 4 |
| trans-2-Butene | 12 |
| n-Butane | 72 |
| Isobutane | traces |
| 2-Methylbutane | traces |

For the hydroformylation, the feed mixture was passed into the reactor together with synthesis gas (molar ratio of synthesis gas to feed mixture=3.5:1) at a total mass flow of 12.8 g/h. The hydroformylation was carried out at a temperature of 120-130° C. and a pressure of 10-17 bar. The total conversion of butenes (i.e. the conversion of all butenes present in the feed mixture) and the n/iso selectivity (ratio of linear to branched products) was determined by gas chromatography via the product composition.

After an experiment duration of 280 hours, total conversion of butenes was 46% and the n/iso selectivity 95%. The proportion of high boilers was max 0.04% by weight.

In the two examples 1 and 2, it was shown to be possible to use commercially available shaped SiC bodies as support.

1. A process for hydroformylating C2 to C8 olefins in a reaction zone in presence of a heterogenized catalyst system, the process comprising:
passing together a gaseous feed mixture containing the C2 to C8 olefins with synthesis gas over a support composed of a porous ceramic material on which a catalyst system, which comprises a metal from group 8 or group 9 of the periodic table of the elements, at least one organic phosphorus-containing ligand and a stabilizer, is present in a heterogenized form; and
wherein the support has a bed of shaped SiC bodies in which a proportion of pores having a pore diameter in a range between 10 nm to 1000 nm, based on a total amount of pores in the shaped SiC bodies, is 20 to 70%.

2. The process according to claim 1, wherein the shaped SiC bodies do not have a washcoat applied, but are employed without washcoat.

3. The process according to claim 1, wherein the at least one organic phosphorus-containing ligand of the heterogenized catalyst system has a general formula (II) 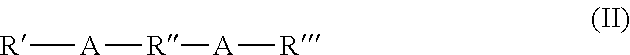
where R′, R″ and R′″ are each organic radicals, with proviso that R′ and R′″ are nonidentical, and both A are each a bridging —O—P(—O)₂ group, wherein two of the three oxygen atoms —O— are attached respectively to radical R′ and to radical R′″.

4. The process according to claim 1, wherein the stabilizer is an organic amine compound containing at least one 2,2,6,6-tetramethylpiperidine unit of formula (I):

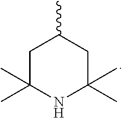

5. The process according to claim 1, comprising:
carrying out the hydroformylation at a temperature in a range from 65 to 200° C.

6. The process according to claim 1, comprising:
hydroformulating at a pressure not greater than 35 bar.

7. The process according to claim 1, wherein the heterogenized catalyst system does not contain any ionic liquid.

8. The process according to claim 1, comprising:
employing C4 olefins in the hydroformylation process.

9. The process according to claim 1, wherein the employed metal from group 8 or 9 of the periodic table of the elements is rhodium.

10. The process according to claim 1, wherein the shaped SiC bodies are from 0.1 mm to 50 mm in size.
